(19) United States
(12) Patent Application Publication
(10) Pub. No.: US 2010/0234329 A1
(43) Pub. Date: Sep. 16, 2010

(54) INGESTIBLE FILM COMPOSITION

(76) Inventors: Eve Bélanger, Ribaute Les Tavernes (FR); Nicole Fortier, St. Hyacinthe (CA); Geneviève Nadeau, Mont St-Hilaire (CA)

Correspondence Address: Michael Ye;Andrews Kurth, 1350 I Street N W, Suite 1100, Washington, DC, 20005, US

(21) Appl. No.: 12/734,419

(22) PCT Filed: Oct. 31, 2008
(86) PCT No.: PCT/CA2008/001917

§ 371 Date: Apr. 30, 2010

Related U.S. Application Data
(60) Provisional application No. 60/984,134, filed on Oct. 31, 2007.

(51) Int. Cl. A61K 47/38; A61K 31/593; A61K 31/437; A61K 31/202; A61K 31/04; A61K 47/36

(52) U.S. Cl. 514/167; 514/299; 514/560; 514/727; 514/778; 514/781

(57) ABSTRACT

The present invention relates to a novel ingestible film composition. More specifically, the invention is directed to a water-based, enzymatically-digested carboxymethylcellulose (CMC-Enz) film composition that is suitable for delivering pharmaceutical drugs, vitamins, natural products and other products to humans and animals. The invention further includes a method for manufacturing the novel composition. Advantageously, the film composition can accommodate an active ingredient in a quantity of up to approximately 60% of the overall weight of the final film.

### CROSS REFERENCE TO RELATED PATENT APPLICATIONS

This application claims the benefit of U.S. Provisional Patent Application No. 60/984,134 filed on Oct. 31, 2007, which is hereby incorporated by reference in its entirety.

### FIELD OF THE INVENTION

The present invention relates generally to the field of dissolving films. More specifically, the invention is directed to a water-based, enzymatically-digested carboxymethylcellulose. (CMC-Enz) film composition that is suitable for delivering pharmaceutical drugs, vitamins, natural products and other products to humans and animals, and includes a method for manufacturing same.

### BACKGROUND OF THE INVENTION

Edible films, such as those found in Listerine PocketPacks™, are known in the art. Other examples of products including such films are Neo Citran® Thin Strips™ for cough (with 25 mg of diphenhydramine hydrochloride) and Gas-X® Thin Strips™ (with 62.5 mg of simethicone) useful for the relief of multiple gas symptoms.

Such films are produced upon drying a wet mix or liquid composition. Advantages to using film strips to deliver drugs and other active ingredients include the following:
Compliance/Swallowing: Eliminates the need for water and the necessity to swallow a bulky pill. It is well-known that many people have difficulty with or are averse to swallowing pills.
Portability/Convenience: Flat packaging available for film strip products is highly portable and excellent for use on-the-go.
Perceived or Actual Faster Onset of Action: Drugs that dissolve rapidly in the mouth can give the perception that the drug acts faster than a traditional pill swallowed in the traditional way. Drugs that dissolve in the mouth have an opportunity to enter the bloodstream more directly via the oromucosal cavity, i.e., they do not have to be absorbed by the digestive system and travel to the liver first before systemic distribution.

While pullulan as a material has been available for quite some time (U.S. Pat. No. 3,784,390 is an example), the use of pullulan with edible food strips has only recently been the subject of patents. See, for example, U.S. Pat. No. 6,887,307. Other materials that are used in the art of edible film strips include cellulose and derivatives such as methylcellulose, sodium alginate, pectin, and Arabic gum to which plasticizers, flavors and other ingredients can be added to create edible film strips with desired properties.

With Na alginate, for example, the maximum load of a drug in a film strip is approximately 10 mg in a 60 mg film strip. Physical limitations that affect the quantity of drug load include the mechanical properties of the film strip, including its dissolution rate, flexibility, tensile strength, tendency to curl and stickiness. Of these, dissolution is perhaps the most important property as far as consumer acceptance and use are concerned. A film should dissolve quickly (i.e., between 10 and 50 seconds) and not leave any residue in the mouth.

While a drug load of 60 mg is adequate for many drugs, a large number of drugs have dosages that range from 80 mg to 150 mg or higher. It would therefore be a significant advantage to have a film strip material that would dissolve more rapidly than Na alginate or non-modified cellulose while maintaining its structural integrity, that can be easily manufactured and that can be made from edible materials which pose no threat to human or animal health. Such a film strip would allow a higher drug load than cellulose-based films and allow new drugs to be formulated with film strip technology.

In addition, several drugs are unstable in high heat, including nitroglycerine, which has been used to treat angina or chest pain for over 50 years. Most drugs degrade more rapidly in the presence of heat, which reduces their ultimate shelf life. Manufacturing these heat-labile or unstable drugs cannot be done at high temperatures. Unfortunately, the drying of films on a commercial scale has typically required use of high temperatures because there is generally the need to evaporate a large amount of water in the slurry in order to dry the film. If less water is used, the slurry becomes too viscous and sticky, making it unsuitable for use in many applications.

It is known by those of skill in the art that the percentage of solids in the slurry, without significant reduction of the ability to mix and handle the slurry, is usually not greater than about 25% for commercial production. Thus, the temperatures used to dry films must be elevated in order for all the water to be evaporated from the film. A possible solution to this problem for conventional materials such as pullulan is to lower the temperature and extend the drying time. This has been done with excellent mechanical results but this process may require several days of drying and is not commercially feasible. (See Kawahara, M., et al., Dependence of the Mechanical Properties of a Pullulan Film on the Preparation Temperature, Biosci. Biotechnol. Biochem. 67:893-895 (2003).) Another option is to use solvents that are more volatile than water, such as ethanol, to enhance evaporation and lower processing temperatures, but there are several disadvantages with this: 1) water-soluble films are generally insoluble in volatile liquids; 2) volatile liquids generally pose safety hazards to the operators and questions about the safety of the resulting film strips; and 3) disposal of volatile liquids after commercial production is cumbersome. For all these reasons, the use of water in commercial manufacturing processes remains the solvent of choice.

A film material that would allow the use of water as a solvent and a higher proportion of solids in the slurry compared with currently used materials without compromising viscosity or drying time would allow the use of a lower temperature during processing and be of significant advantage in the development of film strip formulations for many heat-sensitive drugs. United States Patent Publication Nos. US 2007/0098779 and US 2007/0166371, both belonging to Hanzen et al., describe films and capsules made from modified carboxymethylcellulose materials that are produced with lower amounts of water and that are quickly dissolving. However, such films do not appear to be suitable for active ingredients that are water insoluble. In addition, the films described by Hanzen et al. hold similar amounts of an active ingredient compared to other film strips made of Na alginate or non-modified cellulose, which is not suitable for all active ingredients that would benefit from an orally dissolvable delivery method.

There is thus a need for thin film strip that is easy to produce from a composition having a low solvent content and that can contain active ingredients that are water insoluble. Ideally, such a film will be flexible and resistant to breakage, have an enhanced active load and dissolve quickly with little water. The present invention seeks to meet all of these needs.

### SUMMARY OF THE INVENTION

The invention relates to a physiologically ingestible film composition that is suitable for rapid dissolution in the mouth of a human or animal. The invention further includes a method of making such a film.

Since enzymatically-digested carboxymethylcellulose (CMC-Enz) dissolves faster in the mouth than Na alginate or other polymers, a film strip made from CMC-Enz can be manufactured with a greater thickness and thus contain a greater drug quantity, yet dissolve with a rate similar to that of a Na alginate-based film strip. CMC-Enz also allows for a higher percentage of active ingredients relative to the total weight of the film strip compared to other polymers.

The composition of the present invention is characterized by the inclusion of a CMC-Enz, such as Cekol® EH01 (available from Cp Kelco). CMC-Enz is a water soluble dietary fibre with a lower molecular mass range than that of sodium carboxymethyl cellulose itself. It is prepared from regular food-grade carboxymethylcellulose by partial enzymatic hydrolysis under mildly acidic conditions with a food-grade cellulase enzyme (e.g., from Trichoderma sp. T. longibrachiatum). Solutions of CMC-Enz have a lower viscosity than solutions containing an equal amount of nonmodified carboxymethyl cellulose. This lower viscosity allows for the preferable properties of film strips containing CMC-Enz. CMC-Enz also acts as a stabilizer with fat-extending properties.

Advantageously, Cekol® EH01 can be dissolved in hot or cold water at concentrations of up to approximately 35% by weight. Since a lower quantity of solvent is added, the drying time of the films produced with CMC-Enz are greatly reduced. In addition, films with heat-labile flavors and actives can be manufactured with less damage during processing.

Use of a CMC-Enz of this type (or having similar properties) results in the creation of a solution that has a high solid content and a low viscosity, which makes it suitable for the creation of a thin film. Advantageously, the resultant film can accommodate an active ingredient in a quantity of up to approximately 60% of the overall weight of the final film. This represents a significant improvement over currently available oral films.

Other features of the novel composition described herein are the following:
It is fast dissolving;
It leaves no residue in the mouth;
Being a water-based system, there is no need for the use of alcohol or other solvents during its production;
It can hold both water soluble and water insoluble active ingredients;
It can be dried at low temperatures to produce a film; and
It can be packaged in accordance with methods known in the art.

From a manufacturing standpoint, the fact that the film can be dried at low temperatures from sources such as hot air allows for greater production opportunities. Drugs that are heat-sensitive or labile can be destroyed in part or even in whole during production. Consequently, the film of the present invention offers an alternative that may not heretofore have been available to drug manufacturers.

Advantageously, it has been found that the composition of the present invention may include active ingredients that both water soluble and water insoluble (such as nitroglycerin). The incorporation of emulsifiers such as lecithin and polysorbate 80 into the film composition allows for both types of active ingredients to be used. This represents an improvement over previously developed films.

In addition, emulsifiers enhance the quality of the film that is produced from the composition. The use of emulsifiers results in a film that is smooth rather than glassy. It further enhances the physical characteristics of the final film product by making it less vulnerable to breakage into small pieces, and allows the dried film strip to be lifted from the polyester membrane after drying.

In an embodiment, the film is composed of a combination of one or more of the following ingredients: a film former, a solvent, an emulsifier, a plasticizer, a thickener, a humectant, an alkalizing agent, flavoring and sweetening agents, coloring agents, and an active ingredient. A more specific example of a film having such ingredients would be one comprised of the following constituents: CMC-Enz, water, lecithin, Na alginate, polysorbate 80, sorbitol, glycerol, modified corn starch, propylene glycol, maltodextrin, sucralose, acesulfame K and flavoring.

Depending on the application, the resultant film may be used for the oral administration of an active ingredient (either a drug or a non drug). Ingestion would occur by placing a film strip in the oral cavity of a human or animal. Advantageously, ingestion in this manner is simplified when compared with more traditional tablets; the film dissolves readily without a liquid and is easy to swallow.

Other objects, advantages and features of the present invention will become more apparent upon reading of the following non restrictive description of preferred embodiments thereof, given by way of example only with reference to the accompanying drawings.

### DESCRIPTION OF THE PREFERRED EMBODIMENTS

Terms and Definitions

The terminology used herein is for the purpose of describing particular embodiments only and is not intended to limit the scope of the present invention.

Use of the singular forms “a,” “an,” and “the” include plural references unless the context clearly dictates otherwise. Thus, for example, reference to “a drug” includes a plurality of drugs.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

Where a value is explicitly recited, it is to be understood that values which are about the same quantity or amount as the recited value are also within the scope of the invention, as are ranges based thereon.

Unless defined otherwise or the context clearly dictates otherwise, all technical and scientific terms used herein have the same meaning as commonly understood by one of ordinary skill in the art to which this invention belongs.

The expressions “active” and “active ingredient”, whether in their singular or plural forms, are used, depending on the context, to mean “drug” or “non drug”. They are used to distinguish the “active” in the film composition from the ingredients that make up the film. The term “animal” means any marsupial, reptile, amphibian or non-human mammal.

For the purposes of the present application, the word “drug” means any substance, whether synthetic or non-synthetic, that is taken primarily for non-dietary needs and usually to treat, cure, prevent or diagnose a disease or to otherwise enhance physical or mental well-being. Drugs include, without limitation, any of the following: pharmaceutical compositions, synthetic or naturally-derived pharmaceutically-active molecules, vaccines, proteins, peptides, nucleotides, hormones, vitamins, minerals and herbal remedies.

In contrast to this, the expression “non drug” is meant to signify an ingredient that is not a drug. Compositions of the present invention may be destined for uses other than to treat, cure, prevent or diagnose a disease. Without limitation, such compositions would include dietary supplements, natural products as well as nutraceutical, cosmeceutical, nutricosmetic and cosmetic compositions.

The term “film” is meant to signify the final product of the present invention that results from spreading and drying (from heating or otherwise) the wet mix that results from combining the different ingredients and constituents that are used to make the novel composition of the present invention. The film is typically very thin—approximately 2.0 to 12.0 mil (1 mil=one thousandth of an inch)—and either clear or opaque.

The term “wet mix” for purposes of this application refers to the state of the mixture that results from combining the different ingredients and constituents that are used to make the novel composition of the present invention. The wet mix normally has a consistency that is intermediate between the liquid and solid states.

Film strips derived from the present invention may contain amounts of an active ingredient between 0.1 to about 1000 mg. Guidance as to particular dosages and methods of delivery is provided in the literature and generally available to practitioners in the art. Although methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

Experimental

The novel film composition of the present invention comprises the following ingredients:
Film former(s): key ingredient(s) that are used to make the film. A CMC-Enz having properties along the lines of Cekol EH01 is the most important one for purposes of the present invention, allowing solids in the film to reach a quantity of approximately 65% by weight. Advantageously, a high quantity of solids will decrease the need for high temperatures while drying the film. Non-limiting examples of film formers are sodium alginate (Na alginate), propylene glycol alginate and pullulan. This type of film former helps the film to remain supple or pliable and slightly sticky, allowing for its eventual packaging.
Solvent: used for the preparation of the liquid phase. The principal solvent used is deionized water. Non-limiting examples of other solvents that could be used in minor quantities include propylene glycol and alcohol.
Emulsifier(s): used to enhance the, texture of the film. Non-limiting examples include lecithin and polysorbate (such as polysorbate 80) and equivalents. Emulsifiers are also used to maintain the flavor and oil soluble actives in a homogeneous state while they are in liquid form. Additionally, the use of an emulsifier allows the dried film strip to be lifted from the polyester membrane after drying.
Plasticizer(s): used to give the film its film-like quality. Non-limiting examples include sorbitol, glycerol, medium chain triglycerides and possibly propylene glycol. They help to keep the film flexible and easy to handle. The quantity of plasticizer must be carefully adjusted; if it is insufficient, the film will be fragile and break, and if it is too great, the resulting film will be sticky.
Thickener(s): used to make the film more substantial. Non-limiting examples include modified corn starch, maltodextrin and mixtures thereof.
Humectant(s): used to keep the film humid. Examples of suitable humectants include propylene glycol and medium chain triglycerides.
Alkalizing agent(s): used to adjust the pH of the composition. Sodium bicarbonate has been found to be useful in many film compositions. Other alkalizing agents are known in the art.
Flavoring agent(s): used to give the film a palatable taste. There are endless sources of flavoring agents available and new ones are constantly being developed.
Sweetening agent(s) (or sweetener(s)): like the flavoring agents, they are used to make the film palatable. These include without limitation sucralose and acesulfame potassium (acesulfame K).
Coloring agent(s): a food-grade substance added to the wet mix to provide a suitable color to the resulting film. These include without limitation FD&C Blue No. 1 and 2, tartrazine, and caramel.
Active ingredient(s): one or more drug(s) or non drug(s) introduced in the film composition. This will depend on the ultimate application for the film. The possibilities are endless.

In an embodiment of the invention, the film comprises ingredients in the approximate proportions indicated in Table 1.

TABLE 1
General Composition of Film Ingredients

| | Approximate Percentage (% w/w) |
|---|---|
| Ingredients | in wet mix |
| Solvent | 45.0-60.0 |
| Emulsifier | 1-3.50 |
| Film Former(s) | 12.0-25.0 |
| Plasticizer(s) | 7.5-25.0 |

NB: A second polymer is required for many applications in order to make a desirable embodiment of the invention.

Optionally, thickeners, humectants, alkalizing agents, flavoring agents, sweetening agents, and active ingredients may be added in varying amounts.

Total of solids: approximately 20% to 60%

Process for Making Film

The ingredients or constituents used to make the composition of the present invention are generally mixed in the following manner. It is important to appreciate that the addition of an active ingredient can occur at different steps depending on its solubility. It is also possible for one of skill in the art to modify the steps and/or combine some of them (such as combining Steps 3 and 4 or combining Steps 5 and 6, for example). Consequently, active ingredients such as caffeine and bioferrin, which are soluble in water, may conveniently be added at different steps during the manufacturing process, whereas active ingredients such as nitroglycerin, which are water insoluble, are more suitably included towards the end of the process.

The active ingredients may be added during Step 2, Step 3, and/or Step 9, for instance, depending on the quantity and properties (water soluble or insoluble, heat sensitive or insensitive) of the ingredient. Preferably, active ingredients that are either water insoluble or heat sensitive, or both, are added later in the process (Step 9). Active ingredients that are heat insensitive and/or highly water soluble may be added earlier in the process (Step 2).
Step 1: Addition of water to reaction vessel.
Step 2: Addition of sweeteners (such as sucralose and acesulfame-K) to reaction vessel.
Step, 3: Addition of emulsifier (for instance, lecithin) to reaction vessel.
Step 4: First pre-mix of ingredients (for instance, plasticizers such as glycerol and film formers such as Na alginate).
Step 5: Introduction of film former, such as CMC-Enz (Cekol® EH01 and pullulan if needed) in the reaction vessel. (The use of a funnel can simplify things, given the quantity of CMC-Enz that is added.)
Step 6: Second pre-mix of ingredients (for instance thickeners, such as starch and maltodextrin), performed under vacuum and with rotating knife in bottom of reaction vessel.
Step 7: Introduction of an emulsifier such as polysorbate 80 in the reaction vessel.
Step 8: Optionally, pasteurization is performed by increasing the temperature up to approximately 60° C., but not more than 65° C., to prevent bacterial contamination. The vessel is then cooled to approximately 40° C.
Step 9 Addition of humectant, if required.
Step 10: Introduction of flavoring agent.
Step 11: Introduction of alkalizing agent, if required.
Step 12: Introduction of plasticizer, such as propylene glycol.

NB: Some of the above steps may be performed under vacuum in order to prevent the appearance of bubbles in the final film. Other suitable means for removing bubbles are known in the art (such as the use of a deaerator) and may alternatively be used in applications where bubbles are undesirable.

Wet Mix

Significantly, no heating is required during the mixing. Friction due to viscosity may cause hₑₐt to rise and therefore cooling may be required. The temperature should be regulated so that it does not exceed 65° C. (or as specified by the active ingredient). There is no need to cool the solution down before application of the liquid solution on the polyester membrane (such as Mylar®) used for drying the film (see below). The water temperature will be no lower than 4° C. An ideal temperature for water for many applications will be about 20° C.±5° C.

Drying

The liquid mixture is applied to a polyester membrane (such as Mylar®) or similar surface using a knife over a roll system. The film is then dried in an oven by blowing hot air over its top surface or across both of its surfaces simultaneously. The film is next rolled onto itself, keeping the polyester membrane layer in place.

The following general steps outline a possible drying process to make a film in accordance with the present invention:

1. The wet mix composition is stored into a container;

2. The wet mix is pumped to the front of the drying apparatus;

3. The wet mix is settled onto a polyester membrane and spread to form a film;

4. The film is carried through the heating zones with a speed of 9 to 15 ft/min;

5. The film is heated and dried by hot air flow at approximately 130° C. to 190° C.;

6. The dried film is rolled; and

7. The roll is wrapped to preserve humidity and prevent contamination.

Means other than hot air are possible for drying the film and are within the purview of a person of skill in the art, including without limitation the use of infrared technology and stainless steel conveyor belts.

Packaging

In keeping with manufacturing practices, the roll of film produced above is sliced and then cut into rectangular or square shapes to be placed in a plastic or foil pouch and sealed to create single or multiple doses of the drug.

A possible packaging process for single dosage films is as follows. The dried film, which is wrapped in a roll with polyester backing is slit, cut and packaged the final strip, as described below:

1. The film and polyester backing are separated mechanically;

2. Knives are used to cut the film at regular intervals;

3. The resulting film is settled onto a piece of foil;

4. Another piece of foil comes over the film strip and foil of step 3; and

5. The foil with the film strip inside is sealed and cut to form a single pouch.

The final film humidity will be approximately 2%-20%.

### Example 1

### Caffeine Film Strip Solution

| | Weight (g) |
|---|---|
| Ingredients | in wet mix |
| Water | 500 |
| Sucralose | 5.25 |
| Acesulfame-K | 1.20 |
| Lecithin | 10.00 |
| Pure Cote B-792 | 100.00 |
| CMC-Enz | 275.00 |
| Glycerol | 30.00 |
| Sodium alginate | 10.00 |
| Polysorbate 80 | 12.00 |
| Flavor | 17.50 |
| Propylene glycol | 55.80 |
| Caffeine | 6.20 |
| Maltodextrin | 100.00 |

Film weight: 53 mg

Film dimension: ⅞ inches×1¼ A inches

Amount of active ingredient per strip: 600 μg

Method for Making Film:

Deionized water must be at 55° C. The ingredients should be stirred (or mixed) constantly as they are combined.

Steps:
1. Sucralose and Acesulfame K are pre-mixed and water is added at room temperature. Everything is thoroughly combined by using a mixer at 200 RPM.
2. Lecithin is added to the mixture and stirred for 4 minutes at 400 RPM.
3. Caffeine is added to the mixture and stirred for 5 minutes at 400 RPM.
4. Glycerin and Na alginate are pre-mixed in order to create a homogeneous mixture. This pre-mixture is added to the basic solution and stirred for 30 minutes at 1000 RPM.
5. CMC-Enz is added to the solution and stirred for 40 minutes at 1000 RPM.
6. A modified corn starch (such as Pure Cote®) and maltodextrin are pre-mixed until the resulting mixture has a uniform color. This pre-mixture is then added to the main solution and mixed for 8 minutes at 1000 RPM.
7. Polysorbate 80 is then added to the main solution and mixed for 4 minutes at 1000 RPM.
8. The solution is pasteurized by heating to 62° C.±2° C. and then cooled to 40° C.±2° C.
9. Propylene glycol is added to the solution and stirred for 15 minutes at 1000 RPM.
10. Flavoring is added and the solution mixed for 15 minutes.
11. The final wet mix is then ready to be spread into a film and packaged.

### Example 2

### Bioferrin Film Strip Solution (Composition 1)

| Ingredients | % (w/w) |
|---|---|
| Water | 58.27 |
| Coloring agent | 0.01 |
| Glycerol | 5.36 |
| CMC-Enz | 14.91 |
| Sucralose | 0.78 |
| Acesulfame-K | 0.18 |
| Lecithin | 1.50 |
| Medium chain triglyceride | 3.13 |
| Flavoring agent | 0.74 |
| Bioferrin | 15.12 |

Film weight: 83 mg

Film dimension: ⅞ inches×1¼ inches

Amount of active ingredient per strip: 30 mg

Method for Making Film:

Deionized water must be at room temperature. The ingredients should be stirred (or mixed) constantly as they are combined.

Steps:
1. Sucrelose and Acesulfame K are pre-mixed and water is added at room temperature. Everything is thoroughly combined by using a mixer at 200 RPM.
2. Coloring agent is added to the mixture and stirred for 1 minute at 200 RPM.
3. Lecithin is added to the mixture and stirred for 4 minutes at 400 RPM.
4. Sorbitol, glycerin and Na alginate are pre-mixed in order to create a homogeneous mixture. This pre-mixture is added to the basic solution and stirred for 30 minutes at 1000 RPM.
5. The solution is heated to 65° C. As soon as the solution reaches 65° C., it is cooled to 35° C. This is an optional pasteurization step.
6. Pullulan, CMC-Enz and bioferrin (from lactoferrin source) are pre-mixed until the resulting mixture has a uniform color. This pre-mixture is then slowly added to the solution at 35° C. Everything is mixed for 45 minutes at 1000 RPM, making sure to maintain the overall temperature of the solution below 40° C.
7. Modified corn starch and maltodextrin are pre-mixed until the resulting mixture has a uniform color. This pre-mixture is then added to the main solution and mixed for 8 minutes at 1000 RPM, making sure once again to maintain the overall temperature of the solution below 40° C.
8. Propylene glycol and medium chain triglycerides are then added in sequence. The resulting solution is mixed for 5 minutes at 1000 RPM while maintaining the overall temperature of the solution below 40° C.
9. Finally, polysorbate 80 and flavoring agent (such as lemon flavoring) are pre-mixed before addition to the main solution. The solution is mixed for 10 minutes while maintaining the overall temperature of the solution below 40° C.
10. The final wet mix is then ready to be spread into a film and packaged.

### Example 3

### Bioferrin Film Strip Solution (Composition 2)

| Ingredients | Approximate % (w/w) |
|---|---|
| Solvent | 45.0-60.0 |
| Sweetening agent | 0.10-0.45 |
| Emulsifier | 0.75-3.50 |
| Film former | 10-25 |
| Plasticizer | 7.5-25.0 |
| Flavoring agent | 0.50-2.50 |
| Humectants | 2.00-7.00 |
| Stabilizer | 6.50-15.00 |
| Coloring agent | 0.01-1.50 |
| Bioferrin | 13.00-45.00 |

### Example 4

### Vitamin D3 400 IU Strip Solution

| Ingredients | Approximate % (w/w) |
|---|---|
| Solvent | 30.0-50.0 |
| Sweetening agent | 0.30-0.55 |
| Emulsifier | 1.00-3.50 |
| Film former | 15.0-25.0 |
| Plasticizer | 3.0-7.0 |
| Flavoring agent | 6.0-9.0 |
| Humectants | 6.0-9.0 |
| Stabilizer | 13.0-17.0 |
| Vitamin D3 | 8.0-12.0 |

### Example 5

### Nitroglycerin Film Strip Solution

| Ingredients | Approximate % (w/w) |
|---|---|
| Solvent | 35.0-55.0 |
| Sweetening agent | 0.40-0.70 |
| Emulsifier | 1.0-3.0 |
| Film former | 20.0-35.0 |
| Flavoring agent | 1.0-3.0 |
| Stabilizer | 15.0-20.0 |
| Nitroglycerin | 5.0-8.0 |

### Example 6

### Omega 3 Fatty Acids Film Strip Solution

| Ingredients | Approximate % (w/w) |
|---|---|
| Solvent | 25.00-45.00 |
| Sweetening agent | 0.30-0.55 |
| Emulsifier | 1.00-3.50 |
| Film formers | 15.00-25.00 |
| Plasticizer | 3.00-6.00 |
| Flavoring agent | 0.50-10.00 |
| Humectants | 3.00-10.0 |
| Stabilizer | 10.00-20.00 |
| Coloring agent | 0.25-2.00 |
| Omega 3 Fatty Acids | 20.00-35.00 |

### Example 7

### Thin Film and Na Alginate Dissolution Comparison Test

For the purposes of the present test, strips of Vitamin D3 400 IU were used. The results clearly demonstrate the significant speed of dissolution of a CMC-Enz film composition according to the present invention when compared with a Na alginate film composition.

Description: An external laboratory was asked to place strips of film produced in accordance with the present invention as well as Na alginate film strips in 10 ml of water at a temperature of 37° C. and mix the solutions at 60 rpm. The results, shown in Table 3, reveal how much faster the CMC-Enz-based film strips dissolve in water.

TABLE 3
Dissolution Times for CMC-Enz and Alginate Films

Formulation:

| | Thin film | Na alginate film |
|---|---|---|
| | formulation | formulation |
| Dissolution time | 17 seconds | 1 min. 08 sec. |
| (6 samples) | 16 seconds | 1 min. 00 sec. |
| | 17 seconds | 1 min. 15 sec. |
| | 18 seconds | 1 min. 00 sec. |
| | 18 seconds | 1 min. 20 sec. |
| | 19 seconds | 1 min. 15 sec. |
| Average: | 18 seconds | 1 min. 10 sec. |

Although the present invention has been described hereinabove by way of preferred embodiments thereof, it can be modified without departing from the spirit, scope and nature of the subject invention, as defined in the appended claims.

1. A film composition comprising a solvent, enzymatically-digested carboxymethylcellulose, an emulsifier and a plasticizer.

2. The film composition as defined in claim 1, further comprising a second film former.

3. The film composition as defined in claim 1, further comprising an active ingredient.

4. (canceled)

5. (canceled)

6. The film composition as defined in claim 1, wherein said emulsifier is lecithin or polysorbate 80.

7. The film composition as defined in claim 1, wherein said plasticizer is sorbitol, glycerol or propylene glycol.

8. The film composition as defined in claim 1, further comprising one or more ingredients chosen from the group consisting of plasticizers, thickeners, humectants, alkalizing agents, flavoring agents, sweetening agents, and coloring agents.

9. The film composition as defined in claim 8, wherein said thickener is modified corn starch, maltodextrin or a combination of the modified corn starch and maltodextrin; said humectant is propylene glycol; said alkalizing agent is sodium bicarbonate; and/or said sweetening agent is sucralose or acesulfame K.

10. (canceled)

11. (canceled)

12. (canceled)

13. A The film composition as defined in claim 8, comprising one or more of acesulfame-K, modified corn starch, sorbitol, glycerol, Na alginate, polysorbate 80, propylene glycol or maltodextrin.

14. (canceled)

15. The film composition as defined in claim 3, wherein said active ingredient is caffeine, bioferrin, Vitamin D3, nitroglycerin or Omega 3 fatty acids.

16. (canceled)

17. (canceled)

18. The film composition as defined in claim 1 which is a thin film.

19. (canceled)

20. A method of making a film comprising mixing a solvent, enzymatically-digested carboxymethylcellulose, an emulsifier and a plasticizer to form a wet mix; and using the resulting wet mix to form a film.

21. The method of making a film as defined in claim 20, further comprising cutting and packaging said film.

22. The method of making a film as defined in claim 20.

23. The method of making a film as defined in claim 20, further comprising the addition of an active ingredient.

24. (canceled)

25. (canceled)

26. The method of making a film as defined in claim 20, wherein said emulsifier is lecithin or polysorbate 80.

27. The method of making a film as defined in claim 20, wherein said plasticizer is sorbitol, glycerol or propylene glycol.

28. The method of making a film as defined in claim 20, further comprising the addition of one or more ingredients chosen from the group consisting of plasticizers, thickeners, humectants, alkalizing agents, flavoring agents, sweetening agents, and coloring agents.

29. The method of making a film as defined in claim 28, wherein said thickener is modified corn starch, maltodextrin or a combination of the modified corn starch and maltodextrin; said alkalizing agent is sodium bicarbonate; and/or said sweetening agent is sucralose or acesulfame K.

30. (canceled)

31. (canceled)

32. The method of making a film as defined in claim 28, comprising addition of one or more of acesulfame-K, modified corn starch, sorbitol, glycerol, Na alginate, polysorbate 80, propylene glycol, or maltodextrin.

33. (canceled)

34. (canceled)

35. The method of making a film as defined in claim 23, wherein said active ingredient is caffeine, bioferrin, Vitamin D3, nitroglycerin or Omega 3 fatty acids.

36. (canceled)

37. (canceled)
